(19) United States
(12) Patent Application Publication
(10) Pub. No.: US 2026/0263170 A1
(43) Pub. Date: Sep. 10, 2026

(54) SITUATIONAL AWARENESS OF SURGICAL ROBOT WITH VARIED ARM POSITIONING

(71) Applicant: AURIS HEALTH, INC., Santa Clara, CA (US)

(72) Inventors: Jacob Childs, Huntington Beach, CA (US); Drake Long, Santa Clara, CA (US); Melissa Stachulski, Novi, MI (US); Akshay Sainag Reddy Puli, San Jose, CA (US)

(21) Appl. No.: 19/661,592

(22) Filed: Apr. 28, 2026

Related U.S. Application Data
(63) Continuation of application No. PCT/IB2024/060889, filed on Nov. 4, 2024.
(60) Provisional application No. 63/596,110, filed on Nov. 3, 2023.

(51) Int. Cl. A61B 34/00; A61B 34/10; A61B 34/30; A61B 34/35

(52) U.S. Cl. CPC A61B 34/25; A61B 34/30; A61B 34/35; A61B 2034/102

(57) ABSTRACT

A surgical robotic system comprises a plurality of robotic arms comprising a first robotic arm, wherein the first robotic arm is coupled to an instrument, and a console communicatively coupled to the robotic arms. The console comprises a display device, and a processor coupled to the display device and configured to display, at the display device, an instrument-to-arm mapping model comprising a graphical representation of the robotic arms, wherein the instrument-to-arm mapping model depicts a position of each of the robotic arms, and indicate, in the instrument-to-arm mapping model, instrument data describing the instrument in association with the first robotic arm.

### PRIORITY

This application is a continuation of International Application No. PCT/IB2024/060889, filed Nov. 4, 2024, entitled “SITUATIONAL AWARENESS OF SURGICAL ROBOT WITH VARIED ARM POSITIONING,” which claims priority to U.S. Provisional Application No. 63/596,110, filed Nov. 3, 2023, entitled “SITUATIONAL AWARENESS OF SURGICAL ROBOT WITH VARIED ARM POSITIONING,” the disclosures of which are incorporated by reference herein, in their entirety.

### TECHNICAL FIELD

The systems and methods disclosed herein are directed to devices and methods for indicating locations or orientations of surgical tools, and more particularly to surgical robotic systems for indicating locations or orientations of surgical instruments.

### BACKGROUND

A robotic system may be useful to perform various tasks and procedures. Robotic systems may be used throughout a variety of different industries, such as manufacturing, automotive, healthcare, construction, etc. For example, in the healthcare industry, robotic surgical systems have been used to perform a vast array of medical procedures, including both minimally invasive procedures (e.g., laparoscopic procedures) and non-invasive procedures, (e.g., endoscopic procedures). In general, robotic systems may include robotic arms configured to control the movement of tools or instruments attached to the robotic arms and a console through which a user may control the movements of the robotic arms and/or tools.

### SUMMARY

In an embodiment, a surgical robotic system is disclosed. The surgical robotic system comprises a plurality of robotic arms comprising a first robotic arm, wherein the first robotic arm is coupled to an instrument, and a console communicatively coupled to the robotic arms. The console comprises a display device, and a processor coupled to the display device and configured to display, at the display device, an instrument-to-arm mapping model comprising a graphical representation of the robotic arms, wherein the instrument-to-arm mapping model depicts a position of each of the robotic arms, and indicate, in the instrument-to-arm mapping model, instrument data describing the instrument in association with the first robotic arm.

In another embodiment, a method performed by a surgical robotic system is disclosed. The method comprises displaying, at a console of the surgical robotic system, an instrument-to-arm mapping model comprising a graphical representation of a plurality of robotic arms of the surgical robotic system, wherein the instrument-to-arm mapping model depicts a position of each of the robotic arms relative to a patient platform of the surgical robotic system, and indicating, in the instrument-to-arm mapping model, instrument data describing an instrument in association with a first robotic arm of the robotic arms, wherein the first robotic arm is coupled to the instrument.

In another embodiment, a non-transitory, computer-readable medium storing instructions is disclosed. The non-transitory, computer-readable medium storing instructions, when executed by a processor of a surgical robotic system comprising a plurality of robotic arms, cause the processor to display, at a console of the surgical robotic system, an instrument-to-arm mapping model comprising a graphical representation of the robotic arms with respect to a patient platform of the surgical robotic system, indicate, in the instrument-to-arm mapping model, instrument data describing an instrument in association with a first robotic arm of the robotic arms, wherein the first robotic arm is coupled to the instrument, and display, in the instrument-to-arm mapping model, a haptic interface device (HID) indicator with the instrument data, wherein the HID indicator indicates whether a first HID or a second HID of the console is configured to control the instrument.

In another embodiment, a surgical robotic system is disclosed. The surgical robotic system comprises a patient platform, a plurality of robotic arms comprising a first robotic arm, wherein the first robotic arm is coupled to an instrument, and a console communicatively coupled to the robotic arms. The console comprises a first haptic interface device (HID) and a second HID, and a display configured to display an instrument-to-arm mapping model comprising a graphical representation of the robotic arms with respect to a patient platform of the surgical robotic system, indicate, in the instrument-to-arm mapping model, instrument data describing an instrument in association with the first robotic arm, and display, in the instrument-to-arm mapping model, a haptic interface device (HID) indicator with the instrument data, wherein the HID indicator indicates whether a first HID or a second HID of the console is configured to control the instrument.

Note that the various examples described above can be combined with any other examples described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates a display at a console of robotic system according to various embodiments of the disclosure.

FIGS. 2A, 2B, 2C, and 2D illustrate examples of an instrument-to-arm mapping model and setting menus that may be presented at a display device of the console shown in FIG. 1 according to various embodiments of the disclosure.

FIG. 3 illustrates an example of a stadium view model that may be presented at a display device of the console shown in FIG. 1 according to various embodiments of the disclosure.

FIG. 4 illustrates an example of a destination screen model that may be presented at a display device of a console shown in FIG. 1 according to various embodiments of the disclosure.

FIG. 5A and B illustrate examples of a status model that may be presented at a display device of the console shown in FIG. 1 according to various embodiments of the disclosure.

FIG. 6 illustrates an example of presenting the various models of FIGS. 2A-C, FIG. 3, FIG. 4, and FIGS. 5A-B at a display device of the console shown in FIG. 1 according to various embodiments of the disclosure.

FIG. 7 is a flowchart illustrating a first method according to an embodiment of the disclosure.

FIG. 8 is a flowchart illustrating a second method according to an embodiment of the disclosure.

FIG. 9 is a schematic diagram illustrating electronic components of a surgical robotic system in accordance with some embodiments.

### DETAILED DESCRIPTION

### A. Robotic Systems

Robotic systems may include various components that work together to perform specific tasks. While the specific design and functionality of the robotic system may vary based on the industry and application, some examples of robotic systems may include one or more robotic arms communicatively coupled to a console. The console may be used to manipulate the robotic arms and instruments detachably attached to the robotic arms. The robotic arm may include one or more joints and links interconnecting the joints. The robotic arm may also include an end effector (e.g., gripper, tool, instrument, etc.) positioned at or detachably attached to the robotic arm, such that the end effector interacts with an environment or subject.

The console of the robotic system may essentially function as a control center or interface, allowing operators or users to interact with and control the robot. The console may serve as a point of command and feedback of the robotic system, providing mechanisms for users to input instructions, monitor the status of various components of the robotic system, and receive information regarding the components and environment of the robotic system. For example, the console may include a display device configured display an image captured by a camera of the robotic system, data describing the components of the robotic system, settings and statuses of the components of the robotic system, menus related to the control of the robotic system, and other data describing the robotic system. The console may also include one or more input devices configured to control the robotic arms/end effectors of the system. The input devices, may include, for example, buttons, switches, touch sensitive surfaces, gimbals, haptic interface devices (HIDs), etc., positioned at various areas of the console (e.g., near the hands of the user or near the feet of the user). The user of the console may actuate or interface with the input devices to control the robotic arms/end effectors, manipulate the information being displayed at the console, adjust the settings of the console, etc. As should be appreciated, robotic systems may include many other components aside from the robotic arms and the console, such as, for example, processing hardware, sensors, power sources, safety systems, programming interfaces, memories, etc.

For illustrative purposes, various embodiments may be described herein with respect to a surgical robotic system used in the medical industry. However, it should be appreciated that the robotic system may apply to numerous other industries, and the robotic system described herein should not be limited to solely the surgical robotic system.

To this end, an example surgical robotic system may include multiple robotic arms, the movement of which may be controlled by HIDs located on a console of the surgical robotic system. The robotic arm may be positioned in a manner that facilitates performance of a procedure on a patient, and a medical provider may manipulate the HIDs to control movement of the end effectors (e.g., which may include medical instruments, cameras, etc.) of the robotic arms, which may interact with or be positioned within the body of the patient. At least one of the instruments may include a camera, which may be used to capture a live image of the patient anatomy during the procedure. The image may also show the movement of the end effectors with respect to the patient anatomy. The display device of the console may display the image in real-time, providing a visual feedback to the medical provider while performing the procedure, which may be crucial to making informed decisions during the procedure.

### B. Display Device of the Robotic System

In some cases, the display device may be embodied as a headset device, in which the structure of the display device essentially envelops an area around the eyes of the user to create a sense of presence and immersion with the image and data displayed by the display device. When the user is immersed, the user may insert his or her head into the display device in such a manner so as to block out ambient light when immersed. In this way, the user is enabled to focus on the image (e.g., the patient anatomy) and the movement of the instruments while performing various tasks (e.g., the procedure on the patient), all of which may be shown in the display device. The user may perform these tasks without being distracted by any movement or occurrences happening outside of immersion. Indeed, immersion at the display device reduces the possibility of the medical provider being distracted during a procedure, which might otherwise result in harm or injury to the patient.

However, immersion at the display device limits the amount of information that the user has access to while perform tasks using the robotic arms. For example, the display device may be limited to showing an image of the patient received by a camera scoping the patient. In this way, the information displayed at the display device may generally be minimized to, again, reduce the risk of distracting the user from the primary focus of the procedure.

In some cases, additional information may also be displayed at the display device, but the information may be limited and non-descriptive of an environment around the robotic arms. For example, the information displayed at the display device may not include a location of each of the robotic arms relative to one another and relative to a subject upon which a task is being performed using the robotic arms (e.g., a patient positioned on a patient platform). This may be because such a display of information may not be spatially conservative, and too much information may distract the user during the procedure.

However, this information regarding the robotic arms and attached instruments may be crucial to making informed decisions during the procedure. For example, a medical provider may need a constant awareness of the spatial orientation of the robotic arms to have a table-side visibility of the surgical robotic system. The term “table-side” may refer to an area encompassing the actual patient platform upon which a patient rests during a procedure, robotic arms, instruments, immediate surroundings, etc. of the surgical robotic system (i.e., excluding the console). Such an awareness of the table-side area of the surgical robotic system may be important to the medical provider when troubleshooting robotic arm and instrument collisions, and orienting positions of the instruments in the workspace. Such an awareness may also be important to the medical provider in understanding how the robotic arms move while the patient platform is being adjusted intra-procedurally, and may also be useful in determining how to grasp instruments during a procedure. In this way, failing to indicate the positioning of the robotic arms and the instruments coupled to each of the robotic arms may be problematic because, at certain points before or during the procedure, the medical provider may need access to this information for procedure set-up, troubleshooting, collision avoidance, etc. However, to gain access to this information, the medical provider may need to remove himself or herself from immersion, leave the console, and physically move to a location of the robotic arms to view the positioning of the robotic arms and the attached instruments. Alternatively, another medical provider or personnel physically present at the table-side may need to verbally or otherwise communicate the positioning of the robotic arms and the attached instruments for the medical provider to have knowledge of this information.

In addition, the information displayed at the display device may not indicate a handedness of the system in an easy to understand format that is also spatially conservative (i.e., minimizes display size at the display device). The term “handedness” may refer to the assignment of control of an instrument (and thus, the associated robotic arm to which the instrument is coupled) to a specific input device at the console. Similarly, the information displayed at the display device may not indicate a difference between the instruments that are actively engaged by the input devices, and the instruments that may be coupled to a robotic arm but not actively engaged by the input devices. An instrument may be assigned to an input device when the instrument is coupled (e.g., locked in a detachably attachable manner) to a robotic arm, but an instrument may only be actively engaged by an input device when actions performed at the input devices actually cause movement or manipulation of the robotic arm and/or the attached instrument.

Failing to indicate the handedness of the robotic system to the user may cause confusion to the user and may cause errors and delays while performing a task or procedure using the robotic arms. For example, the medical provider may need extra time before the procedure to either manually identify the instrument that is controlled by each input device, or manually test the input devices to identify the instrument that moves with the movement of the respective input device. This may especially be the case in surgical robotic systems in which a positioning of the robotic arms may not be indicated in a linear and sequential fashion. The robotic arms may not be indicated in a linear and sequential fashion when, for example, the robotic arms translate around a surgical table such that an order of the robotic arms from one side (i.e., left) to another side (i.e., right) is not always sequential. Therefore, other than the image of the subject and basic information on the instruments or robotic arms, the display device may not display details that may be significant or otherwise helpful to the user while performing the procedure.

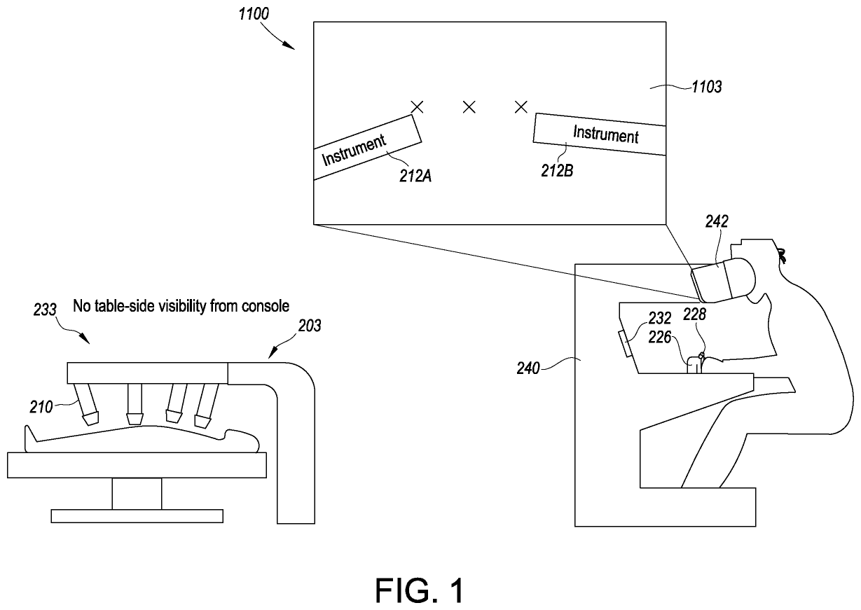

For example, FIG. 1 is a diagram 1100 illustrating the console 240 displaying an image 1103 of a patient anatomy and active instruments 212A, 212B being controlled by a user of the console 240. The console 240 illustrated in FIG. 1 may include a display device 242, a touchscreen 232, and one or more user input devices 226, 228 (also referred to herein as “HIDs 226, 228”). While the HIDs 226, 228 are shown in FIG. 1 as handles or joysticks, it should be appreciated that the HIDs 226, 228 may be embodied as any other type of input device (e.g., button, switch, touch-sensitive surfaces, toggles, etc.)

The console 240 may include the display device 242, a left HID 226 (also referred to herein as a “first HID 226), and a right HID 228 (also referred to herein as a “second HID 228). However, it should be appreciated that the console 240 may include other components that are not otherwise shown or described in FIG. 1.

A medical provider may be seated at the console 240 with his or her head immersed into the display device 242. A left hand of the medical provider may operate the left HID 226, and a right hand of the medical provider may operate the right HID 228. Each of the HIDs 226, 228 may be assigned to control multiple robotic arms 210, but may only be permitted to engage with and actively control one robotic arm 210 at a time, and thus one instrument 212A, 212B at a time. The robotic arms 210 and instruments 212A, 212B that are actively engaged with and controlled by the HIDs 226, 228 may be referred to herein as “active robotic arms 210” and “active instruments 212A, 212B.”

The display device 242 may include a user interface displaying the image 1103. The image 1103 may be an image of a patient anatomy captured by camera, such as, for example, an endoscopic camera or a laparoscopic camera. In one case, the camera 606 may be coupled to one of the robotic arms 210 and capturing the image 1103 as a live-stream while the medical provider uses the surgical robotic system to perform a procedure on the patient. In another case, the camera 606 may have captured the image 1103 prior to performing a procedure on the patient using the surgical robotic system.

The image 1103 may not only display a portion of the patient anatomy, but may in some cases, also display one or more active instruments 212A, 212B that are actively engaged by the HIDs 226 and 228. In the example shown in FIG. 1, a first instrument 212A and a second instrument 212B are displayed in the image 1103. However, it should be appreciated that in other cases, the image 1103 may not display the instruments 212A, 212B at all (e.g., the instruments 212A, 212B may not have moved into the area captured by the image 1103).

FIG. 1 also shows the table-side area 233 of an environment around the surgical robotic system 203. When immersed in the display device 242, the medical provider may not have access to the actual positioning and orientation information regarding the components of the surgical robotic system 203 at the table-side area 233. The table-side area 233 illustrates an example of an environment around the robotic arms 210 of the surgical robotic system 203. For example, the position and orientation information that may not be indicated in the image 1103 may include position and orientation of the robotic arms 210, various links and/or joints of the robotic arms 210, instruments 212A, 212B information, positioning information of the patient platform, draping information, staff positioning information, tower positioning information, lighting positioning information, etc.).

While FIG. 1 relates to a surgical robotic system 203, it should be appreciated that the description above regarding the lack of visibility of the robotic arms 210 when the user of the console 240 is engaged with the display device 242 applies to other types of robotic systems as well. For example, the robotic arms 210 may be located in a warehouse or a factory, and the console 240 may be positioned in a separate area or room away from the robotic arms 210. A similar situation may arise in which the user of the console 240 may not be aware of the connection between the different user input devices on the console 240 and the respectively controlled robotic arms 210 of the robotic system.

### C. Introduction to Models for Display at the Console

The present disclosure provides a technical solution to the foregoing technical problem related to robotic systems and platforms by displaying the positioning of the robotic arms 210, the instruments 212A, 212B (hereinafter occasionally referred to as “instruments 212”) coupled to each of the robotic arms 210, and an indication of the handedness of the system (i.e., indicates the instruments 212A, 212B are actively engaged with and controlled by a respective HID 226, 228) in an instrument-to-arm mapping model displayed at the console. In an embodiment, the instrument-to-arm mapping model may include a graphical representation of the robotic arms 210 and a current position and orientation of each of the robotic arms 210 relative to a patient platform. The instrument-to-arm mapping model may also include instrument data describing the instruments 212A, 212B coupled to each of the robotic arms 210. The instrument data of an instrument 212A or 212B may be shown in the instrument-to-arm mapping model as being associated with that robotic arm 210.

In some embodiments, a stadium view model of the surgical robotic system 203 may also be displayed at the console 240 in which the stadium view model provides a more holistic view of the surgical robotic system 203. The stadium view model may be a graphical representation or rendering of an area around a subject (e.g., patient) of the robotic system. For example, the stadium view model may display a rendering of a patient, a patient platform upon which the patient rests, and the robotic arms 210 of the surgical robotic system 203. The stadium view model may also display other external individuals, staff, or components in the operating room. In some cases, the stadium view model may display the current positions of the patient, patient platform, and robotic arms 210, and the positions may be updated as these positions change over time. A user may interact with the console 240 (e.g., a touchscreen 232) to display different views of the stadium view model.

In some embodiments, a status model of the surgical robotic system may also be displayed at the console 240. The status model may display the current position and orientation of each link and joint on each of the robotic arms 210 and indicate a status of each link and/or joint on each of the robotic arms 210 (e.g., a certain color/shade may indicate that a link on the robotic arm 210 is locked or unlocked, a certain color/shade may indicate that the robotic arm 210 is coupled to an instrument 212A or 212B and docked to the patient, etc.).

The instrument-to-arm mapping model, stadium view model, and status model may each be two dimensional (2D) or three dimensional (3D) renderings (i.e., not an actual camera view) of various portions of the robotic system, and may include other icons, text, or graphics as described herein. The instrument-to-arm mapping model, stadium view model, and status model may be displayed at the display device 242 and/or the touchscreen 232. When the instrument-to-arm mapping model, stadium view model, or status model is displayed at the display device 242, the model may be displayed in a picture-in-picture (PIP) format, and positioned at a corner of the display device 242, so as to only overlap a small portion of the image of the subject being predominately displayed at the display device 242. When the instrument-to-arm mapping model, stadium view model, or status model is displayed at the touchscreen 232, the model may encompass any portion of the display of the touchscreen 232 (i.e., the display size may not be limited when displayed at the touchscreen 232 since the touchscreen 232 may not be displaying the image of the subject).

In this way, the embodiments disclosed herein provide several advantages to a medical provider and a patient when operating a surgical robotic system. For example, by providing the instrument-to-arm mapping model, stadium view model, or status model to the medical provider at the console 240, the medical provider is enabled to set up the surgical robotic system 203 for the procedure in a far more efficient and effective manner (e.g., the medical provider may quickly obtain the information needed to setup the robotic arms using the models). In addition, the medical provider may also use the instrument-to-arm mapping model, stadium view model, or status model to troubleshoot robotic arms 210 and instruments 212A, 212B collisions, and act accordingly, to prevent injury to the patient and ensure a safe procedure. Lastly, the medical provider may also use the instrument-to-arm mapping model, stadium view model, or status model to understand how the robotic arms 210 move while the patient platform is being adjusted intra-procedurally, which may be useful in making decisions during the procedure and even grasp instruments 212A, 212B during the procedure.

### D. Instrument-to-Arm Mapping Model

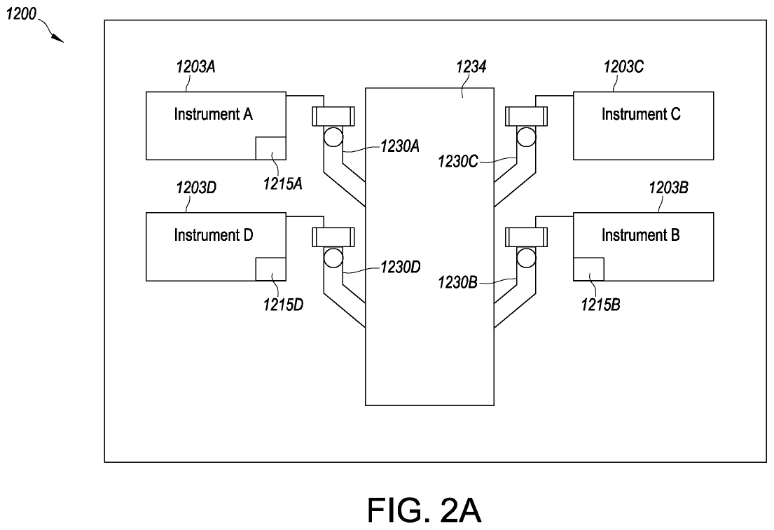

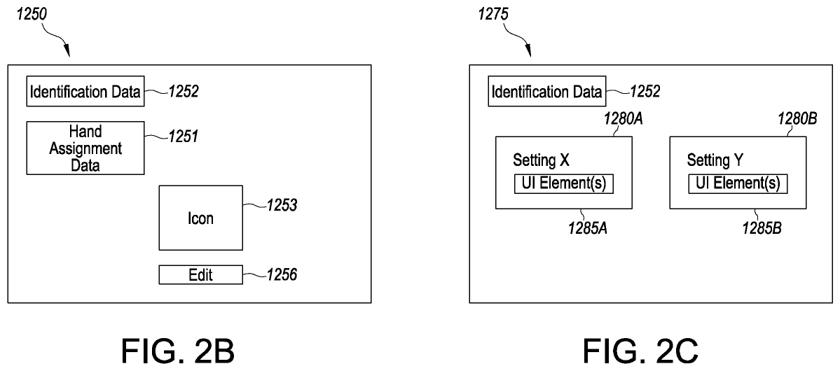

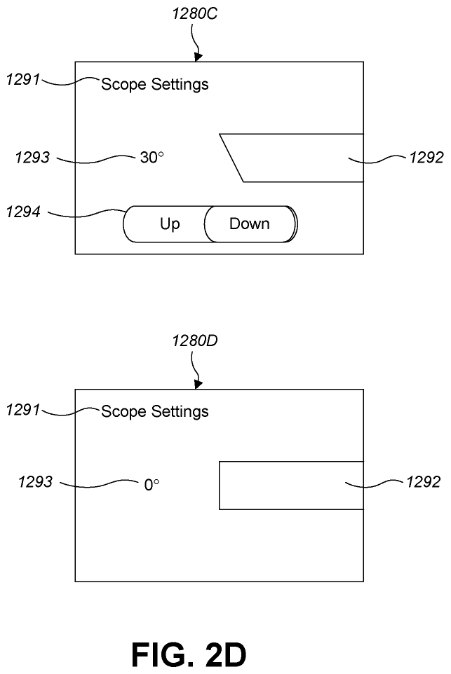

Turning now to FIGS. 2A-2D, shown are various examples of an instrument-to-arm mapping model 1200 and setting menus 1250, 1275 that may be displayed in response to a selection of an indicator in the instrument-to-arm mapping model 1200. Referring specifically now to FIG. 2A, shown is an instrument-to-arm mapping model 1200 according to various embodiments of the disclosure. The instrument-to-arm mapping model 1200 may provide a technical solution to the foregoing technical problem by displaying the additional information at the console 240, such that the user may not have to leave the console 240 to gain access to this information or wait to receive this information from staff near the physical robotic arms 210. In an embodiment, the instrument-to-arm mapping model 1200 may include a graphical representation (or rendering) of each of the robotic arms 210 of the system. In an embodiment, the instrument-to-arm mapping model 1200 may include a graphical representation of each of the joints and/or links of each of the robotic arms 210 of the system. The instrument-to-arm mapping model 1200 may indicate a current position and orientation of each of the robotic arms 210 (and each link/joint along the robotic arm 210), which may be updated as the position and orientation of each of the robotic arms 210 changes during set-up or a procedure.

As shown in FIG. 2A, the instrument-to-arm mapping model 1200 includes a graphical representation of four robotic arms 1230A, 1230B, 1230C, and 1230D (also referred to herein as simply “robotic arms 1230A, 1230B, 1230C, and 1230D”). While four robotic arms 1230A-1230D are shown in this example, it should be understood that any number of arms may be represented. The graphical representation of these four robotic arms 1230A-1230D may be rendered to represent the actual physical structure of four robotic arms 210 deployed by a subject upon which tasks or procedures are performed. The graphical representation of these four robotic arms 1230A-1230D may be rendered in real-time using a rendering application or may be obtained from a library pre-loaded with the graphical representations of the robotic arms 1230A-1230D.

The robotic arms 1230A-1230D may also depict (e.g., include a rendering of) one or more joints and/or links positioned along a corresponding robotic arm 210. The graphical representation of the joints, links, and other components on the robotic arm 210 may be rendered in real-time using a rendering application or may be obtained from a library pre-loaded with the graphical representations of the joints, links, and other components.

In some embodiments, the positions and orientations of each of the robotic arms 1230A-1230D in the instrument-to-arm mapping model 1200 may reflect a current position and orientation of each of the corresponding robotic arms 210. For example, the links and/or joints along each of the robotic arms 210 may include one or more processors or encoders, which may obtain (e.g., compute) position data within the workspace and/or relative to a subject or platform. The links and/or joints along each of the robotic arms 210 may transmit this data to a processor located at or coupled to the console 240. The processor may render robotic arms 1230A-1230D within the instrument-to-arm mapping model 1200 so as to reflect the current and accurate positions and orientations of the robotic arms 210 based on the received position data. The position data may be collected in real-time (i.e., constantly throughout the use of the robotic arms 210), or may be collected based on a pre-defined schedule (e.g., every millisecond (ms), every 2 ms, etc.). In this way, the processor may constantly receive updates describing the positions and orientations of each of the robotic arms 210 with respect to a subject or platform (e.g., at the table-side). The processor may then use the updates to correspondingly update the positions and orientations of each of the robotic arms 1230A-1230D in the instrument-to-arm mapping model 1200.

For example, the positions and orientations of each of the robotic arms 1230A-1230D may be rendered in the instrument-to-arm mapping model 1200 relative to a graphical representation of a patient platform 1234. The graphical representation of the patient platform 1234 may be rendered to represent the actual physical structure of patient platform of a surgical robotic system 203, upon which a patient may be secured during a procedure.

In some embodiments, the instrument-to-arm mapping model 1200 may also display instrument data related to an instrument 212A or 212B coupled to each of the robotic arms 210. The instrument data may be represented in the instrument-to-arm mapping model 1200 as the instrument indicators 1203A-1203D (or icons) shown in FIG. 2A. Each of the instrument indicators 1203A-1203D may be displayed as being associated with a particular robotic arm 1230A-1230D. For example, the instrument indicators 1203A-1203D may be positioned proximate to the robotic arm 1230A-1230D to which an instrument 212A or 212B described by the instrument indicator 1203A-1203D is coupled. The instrument indicators 1203A-1203D may also or otherwise be indicated as connected to the robotic arms 1230A-1230D to which an instrument 212A or 212B described by the instrument indicator 1203A-1203D is coupled by, for example, call lines. A call line may be a line intercoupling an instrument indicator 1203A-1203D with a robotic arm 1230A-1230D, which may indicate that the instrument 212A or 212B described by the instrument indicator 1203A-1203D is coupled to the robotic arm 210 represented by the robotic arm 1230A-1230D. In an embodiment, the instrument indicators 1203A-1203D may remain stationary (i.e., in the same position within the instrument-to-arm mapping model 1200) even if the corresponding robotic arm 1230A-1230D changes positions within the robotic arm 1230A-1230D. In this case, the call lines may extend or adjust to ensure that the call lines connect the stationary instrument indicators 1203A-1203D with the dynamic, changing position of the robotic arms 1230A-1230D.

As shown in FIG. 2A, each instrument indicator 1203A-1203D may include data, icons, images (e.g., camera images or pre-stored digital rendering models), text, identifiers, and/or other data used to describe and identify a particular instrument 212A, 212B. The instrument indicators 1203A-1203D may include an identifier of the robotic arm 210 to which the instrument 212A, 212B described is coupled, an identification of the instrument 212A or 212B, an image of the instrument 212A or 212B, and/or any other data related to the instruments 212A, 212B or a robotic arm 210 to which the instrument 212A or 212B is coupled. The identifier of the robotic arm 210 may be represented as one or more alphanumeric values identifying a robotic arm 210, and the identifier may also be physically present on the robotic arm 210 itself. The identification of the instrument 212A, 212B may be represented as text describing a name of the instrument 212A or 212B. The image of the instrument 212A or 212B may be a rendered graphical representation of the instrument 212A or 212B, or an image of the instrument 212A or 212B obtained from a camera. The image may be rendered in real-time using a rendering application or may be obtained from a library loaded with rendered graphical representations of various instruments 212A, 212B. As disclosed herein, an instrument 212A or 212B may be a medical instrument, a camera device, a grasper, welding tools, drilling tools, lasers, sensors, fastening tools, or any other type of tool, device, or instrument capable of detachably attaching to a robotic arm 210.

Lastly, one or more of the instrument indicators 1203A-1203D may also include HID indicators 1215A, 1215B, and 1215D, which may indicate a handedness and an engagement status of each of the instruments 212A, 212B described by the respective instrument indicator 1203A-1203D. The HID indicators 1215A-1215B, 1215D may only be included in an instrument indicator 1203A-1203B, 1203D when the instrument 212A, or 212B described is controlled by either the left HID 226 or the right HID 228. As shown in FIG. 2A, only the instrument indicators 1203A-1203B and 1203D include the HID indicators 1215A-1215B and 1215D, and this may be because the robotic arm 210 controlling the instrument 212A or 212B described by the instrument indicator 1203C may not necessarily be assigned to only one of the HIDs 226 and 228. For example, when the instrument 212A, 212B being described by the instrument indicator 1203C is a camera device, the HIDs 226 and 228 may have to operate and move together in a single plane to control movement of the camera device (i.e., a single HID 226, 228 may not be used to control movement of the camera device).

In some embodiments, the HID indicators 1215A-1215B, 1215D may be proximate to or within the respective instrument indicator 1203A-1203B, 1203D. In the example shown in FIG. 2A, the HID indicators 1215A-1215B, 1215D are shown as being positioned within the respective instrument indicators 1203A-1203B, 1203D. However, in other embodiments, the HID indicator 1215A-1215B, 1215D may be positioned outside the respective instrument indicator 1203A-1203B, 1203D, but proximate to, overlapping with, or touching an edge of the respective instrument indicator 1203A-1203B, 1203D. In an embodiment, the HID indicators 1215A-1215B, 1215D may indicate whether the instrument 212A, 212B described is configured to be controlled by the left HID 226 or the right HID 228. For example, the HID indicators 1215A-1215B, 1215D may include text indicating whether the instruments 212A, 212B are configured to be controlled by the left HID 226 (indicated with the text “L”) or the right HID 228 (indicated with the text “R”). Additional icons or graphical representations of hands, for example, may also be positioned within the HID indicators 1215A-1215B, 1215D to easily signal to the user the type of data indicated by the HID indicators 1215A-1215B, 1215D. In one example, if instrument 212A is being actively controlled by the left HID 226 and instrument 212B is being actively controlled by the right HID 228, the HID indicator 1215A of instrument 212A may illustrate an icon of a left hand and the HID indicator 1215B of instrument 212B may illustrate an icon of a right hand.

In some cases, the HIDs 226, 228 may be programmed to control multiple robotic arms 210 and thus multiple, instruments 212A, 212B, but may only be engaged with one instrument 212A, 212B and/or robotic arm 210 at a time. In this way, a HID 226, 228 may actively control one active instrument 212A, 212B, but may be configured to control one or more other inactive instruments 212A, 212B. The HID 226, 228 may switch between active instruments 212A, 212B and inactive instruments 212A, 212B based on user inputs received at user input devices on the console 240 (e.g., pedals at the foot actuator assembly).

To this end, the HID indicators 1215A-1215B, 1215D may indicate whether an instrument 212A, 212B is actively engaged with a HID 226, 228 or is not actively engaged with a HID 226, 228, but is still configured to be controlled by a particular HID 226, 228. The HID indicators 1215A-1215B, 1215D may include, for example, shaded or colored borders around an outer edge of the HID indicators 1215A-1215B, 1215D, which may indicate whether an instrument 212A, 212B is actively engaged or not. For example, the border may be activated or highlighted as a particular color (e.g., blue) when a HID 226, 228 is actively engaged with the instrument 212A, 212B. The border may be de-activated or dulled out to a particular color (e.g., dark grey) when a HID 226, 228 is disengaged with the instrument 212A, 212B. In an example, if an instrument 212 is currently inactive but is available to be controlled by the left HID 226, the HID indicators 1215A-1215B, 1215D may include the text “L” with a circular arrow around the text to illustrate that that instrument 212 is available for swapping control by the left HID 226. Similarly, if an instrument 212 is currently inactive but is available to be controlled by the right HID 228, the HID indicators 1215A-1215B, 1215D may include the text “R” with a circular arrow around the text to illustrate that that the instrument 212 is available for swapping control by the right HID 226.

As should be appreciated, the instrument indicators 1203A-1203D may include other information, images, and/or icons that are not necessarily shown or described herein. In addition, abbreviated versions of the instrument-to-arm mapping model 1200 may be available. For example, an abbreviated version of the instrument-to-arm mapping model 1200 may include only a virtual representation of each instrument 212 to indicate the instrument type without text describing a name of the instrument 212. While only four robotic arms 1230A-1230D and instrument indicators 1203A-1203D are shown in the instrument-to-arm mapping model 1200, any number of robotic arms 1230A-1230D and instrument indicators 1203A-1203D may be included in an instrument-to-arm mapping model 1200 based on the number of robotic arms 210 deployed by the surgical robotic system.

In some embodiments, the instrument indicators 1203A-1203D may be selected by the user, triggering a new menu or window displaying one or more adjustable settings corresponding to the instrument 212 to be displayed at the display device 242. For example, each instrument indicator 1203A-1203D may be an icon, which the medical provider may select, using an input device at the console 240, to open a menu related to instrument 212A, 212B settings. For example, the instrument-to-arm mapping model 1200 may be displayed at the touchscreen 232, and the medical provider may select the instrument indicator 1203A-1203D via the touchscreen interface of the touchscreen 232. By selecting an instrument indicator 1203A-1203D, the medical provider may be attempting to adjust a setting of the related instrument 212A, 212B.

Turning now to FIG. 2B, shown is a setting menu 1250 for an instrument 212A, 212B according to various embodiments of the disclosure. The setting menu 1250 may be displayed after a selection of an instrument indicator 1203A-1203D is received at the console 240. In the example shown in FIG. 2B, the setting menu 1250 may be overlaid on the instrument-to-arm mapping model 1200. In another embodiment, the setting menu 1250 may be displayed as a separate window from the instrument-to-arm mapping model 1200.

The setting menu 1250 may include identification data 1252, which may include an identifier of the robotic arm 210 to which the instrument 212A, 212B is coupled. The identification data 1252 may also include an identification of the instrument 212A, 212B. The identification data 1252 may be in the form of an icon, image, text, or other type of indicator. The setting menu 1250 may also include hand assignment data 1251 indicating which hand of the user may be used to control the instrument 212A, 212B, or which HID 226, 228 is configured to control the instrument 212A, 212B. The hand assignment data 1251 may be text indicating the hand assignment of the instrument 212A, 212B. For example, the hand assignment data 1251 may include the text “Left,” which may indicate that the left hand of the user may be used to control the instrument 212A, and/or that the left HID 226 is configured to control the instrument 212A.

In an embodiment, the setting menu 1250 may also include an icon 1253 depicting the hand assignment of the instrument 212A, 212B. In an embodiment, the icon 1253 may also indicate a finger placement of the assigned hand at the particular HID 226, 228. In an embodiment, the icon 1253 may depict, to the user, the optimal finger positioning of the HID 226, 228. For example, the icon 1253 may show a left hand being positioned around graspers digitally representing the left HID 226. The icon 1253 may be rendered in real-time using a rendering application or may be obtained from a library loaded with graphical representations of different types of HIDs 226, 228 and user engagements with the HIDs 226, 228.

In an embodiment, the setting menu 1250 may also include an edit icon 1256. The medical provider may select the edit icon 1256 to further adjust the settings displayed in the setting menu 1250. For example, another more detailed setting menu may be displayed in response to receiving a selection of the edit icon 1256, in which the hand assignment settings or other settings related to this instrument 212A, 212B or the corresponding robotic arm 210 may be adjusted. It should be appreciated that different types of instruments 212A, 212B may be associated with different types of setting menus 1250.

Referring now to FIG. 2C, shown is another example of a setting menu 1275 according to various embodiments of the disclosure. The setting menu 1275 may be displayed after a selection of an instrument indicator 1203A-1203D is received at the console 240. In the example shown in FIG. 2C, the setting menu 1275 may be overlaid on the instrument-to-arm mapping model 1200. In another embodiment, the setting menu 1275 may be displayed as a separate window from the instrument-to-arm mapping model 1200.

The setting menu 1275 may include identification data 1252, which may include, for example, an identifier of the robotic arm 210 to which an instrument 212A, 212B is coupled. The identification data 1252 may also include an identification of the instrument 212A, 212B. For example, an identification of the instrument 212A, 212B may include text defining a name of the instrument 212A, 212B. The identification data 1252 may also include additional settings of the instrument 212A, 212B, such as, for example, an angle and directional icon indicating an angle and direction of the instrument 212A, 212B.

The setting menu 1275 may include also include one or more setting windows 1280A, 1280B, which may each correspond to a different setting associated with the instruments 212A, 212B being described in the setting menu 1275. For example, when the instrument 212A, 212B is a camera device, the setting window 1280A may indicate settings related to a light on the camera device, and the setting window 1280B may indicate image settings for the camera device. While only two setting windows 1280A, 1280B are shown in the setting menu 1275, it should be appreciated that the setting menu 1275 may include any number of setting windows 1280A, 1280B, each corresponding to a different setting of the instrument 212A, 212B.

Within the setting windows 1280A, 1280B, there may be one or more user interface elements 1285A, 1285B. The user interface elements 1285A, 1285B may indicate a current setting related to the setting being indicated in the respective setting window 1280A, 1280B. For example, the setting window 1280A related to the light on the camera device may include multiple settings, for example, a setting for turning on/off the light, a setting for adjusting the brightness on the light, etc. The user interface elements 1285A may correspond to each of these settings, and may be interacted with by a user via an input device (e.g., touchscreen 232) on the console 240 to adjust the corresponding setting. For example, the interactive elements may be toggle buttons, sliding bars, check boxes, radio buttons, tabs, icons, drag and drop elements, navigation bars, etc. The user interface elements 1285A, 1285B may also include an icon or text indicating the current setting.

Referring now to FIG. 2D, shown are examples of setting windows 1280C-D displayed in a setting menu 1275 according to various embodiments of the disclosure. The setting windows 1280C-D may be displayed after a selection of an instrument indicator 1203A-1203D associated with a scope device or camera is received at the console 240. The setting windows 1280C-D may each display text, images, icons, and/or other user interface elements, each of which may be used to display and/or adjust the settings of the scope device attached to robotic arm 210.

As shown in FIG. 2D, the example setting window 1280C includes text 1291 describing a type of setting indicated in the setting window 1280C. The setting window 1280C may also include a visual representation 1292 of the scope device and corresponding text 1293 indicating an angle of the scope device. The setting window 1280C may also include a user interface element 1294, which when selected or interacted with in a certain manner, may adjust the orientation of the scope device attached to the robotic arm 210.

The graphical representation 1292 of the scope device may be an image or a rendered icon representing the scope device, which may depict the angle and orientation of the scope device. In the example shown in FIG. 2D, the graphical representation 1292 depicts the scope device as being angled down at approximately 30°, the text 1293 states that the scope device is angled at 30°, and the user interface element 1294 indicates that the scope device is angled down. As another illustrative example, when the scope device attached to the robotic arm 210 is angled up at approximately 30°, the graphical representation 1292 may depict the scope device as being angled up at approximately 30°, the text 1293 may state that the scope device is angled at 30°, and the user interface element 1294 may indicate that the scope device is angled up.

The graphical representation 1292 of the scope device may change based on the type of scope device attached to a robotic arm 210. Similarly, the angle of the scope device depicted in the graphical representation 1292 may change based on an actual angle of the scope device. The orientation of the scope device depicted in the graphical representation 1292 may also change based on an actual orientation of the scope device. In the example shown in FIG. 2D, the user interface element 1294 is a toggle user interface element, in which the user may select either the “Up” or the “Down” button on the toggle user interface element to adjust the orientation of the scope device to the selected up/down orientation. When the orientation of the scope device is adjusted based on this user input, the orientation of the scope device depicted in the graphical representation 1292 may also be updated accordingly. It should be appreciated that the user interface element 1294 may be any type of user interface element other than the toggle user interface element shown in FIG. 2D.

The setting menu 1280D is similar to the setting window 1280C in that the setting window 1280D includes text 1291 describing a type of setting indicated in the setting window 1280D, a graphical representation 1292 of the scope device, and text 1293 indicating an angle of the scope device. In particular, the setting window 1280D depicts the settings for a zero-degree scope device, which may have a distal straight end with no angle. To this end, the graphical representation 1292 of the scope device in the setting menu 1280D is depicted as having a straight distal end (i.e., 0°). In this way, the graphical representation 1292 of the scope device may change based on the actual features and settings of the scope device attached to a robotic arm 210 and possibly inside a patient.

### E. Stadium View Model

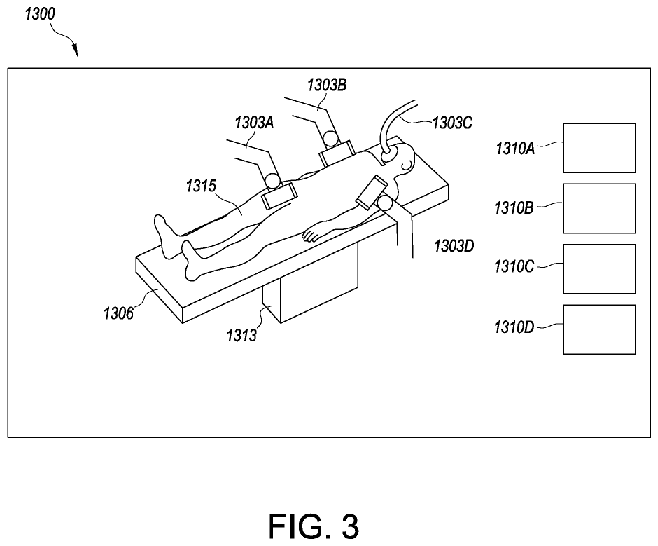

Turning now to FIG. 3, shown is a view of the stadium view model 1300 of a robotic system according to various embodiments of the disclosure. While the robotic system shown in FIG. 3 is a surgical robotic system 203, it should be appreciated that the stadium view model 1300 may be generated for other types of robotic systems throughout various different industries. As mentioned above, the stadium view model 1300 may provide a more holistic view of the patient, patient platform, and the robotic arms 210. For example, the stadium view model 1300 may be a graphical representation or rendering of an environment including not only the entire table-side, but also the patient and/or any other external individuals, staff, or components in the operating room.

The example of the stadium view model 1300 includes a graphical representation of the patient, the patient platform, the robotic arms 210, the base of the patient table, and/or various other structural aspects of the surgical robotic system 203. The stadium view model 1300 may also include different view icons 1310A-1310D that each correspond to different views (or perspectives) of the stadium view model 1300 that may be displayed at the console 240. When different view icons 1310A-1310D are selected by the medical provider, different views of the stadium view model 1300 may be displayed at the console 240. Each of the views may depict the environment around the surgical robotic system from a different perspective (e.g., birds-eye view, high perspective view, low perspective view, side view, etc.).

FIG. 3 shows a first view of the stadium view model 1300 according to various embodiments of the disclosure. The first view may be a first side view from a high perspective, in which a graphical representation of the entire patient, all of the robotic arms 210, the patient platform, and the base of the system is depicted. The first view of the stadium view model 1300 may be displayed with the view icons 1310A-1310D, in which one of the view icons, for example view icon 1310B, corresponds to the first view. In an embodiment, the medical provider may select the view icon 1310B by providing a user input to a user input device of the console 240 (e.g., to a touchscreen interface of the touchscreen 232) to select the view icon 1310B. The selection may then cause the first view of the stadium view model 1300 to be displayed at the console 240. After selection of the view icon 1310B, the view icon 1310B may be highlighted with a different background color (e.g., blue) to indicate that the displayed perspective corresponds to the first view of the stadium view model 1300.

In an embodiment, the stadium view model 1300 may include a graphical representation (or rendering) of each of the robotic arms 210 of the system, and in some embodiments, each of the joints and/or links of each of the robotic arms 210 of the system. In an embodiment, the stadium view model 1300 may indicate a current position and orientation of each of the robotic arms 210, which may be updated as the position and orientation of each of the robotic arms 210 moves during set-up or a procedure. In this way, the stadium view model 1300 may depict a current position and orientation of the robotic arms 210 as deployed at the table-side.

As shown in FIG. 3, the stadium view model 1300 includes a graphical representation of four robotic arms 1303A, 1303B, 1303C, and 1303D (also referred to herein as simply “robotic arms 1303A, 1303B, 1303C, and 1303D”). The graphical representation of these four robotic arms 1303A-1303D may be rendered to represent the actual physical structure of four robotic arms 210 deployed at the table-side of the surgical robotic system 203. The graphical representation of these four robotic arms 1303A-D may be rendered in real-time using a rendering application or may be obtained from a library that may be loaded with the graphical representation of the robotic arms 1303A-1303D.

The robotic arms 1303A-1303D may also depict (e.g., include a rendering of) one or more joints and/or links positioned along a corresponding table-side robotic arm 210. The graphical representation of the joints, links, and other components of the robotic arm 210 may be rendered in real-time using a rendering application or may be obtained from a library that may be loaded with the graphical representation of the joints, links, and other components.

The stadium view model 1300 may also include a graphical representation of the patient platform 1306 and a base 1313 of the surgical robotic system 203. The base 1313 may include different components of the surgical robotic system 203 based on an embodiment of the surgical robotic system 203. As should be appreciated, the stadium view model 1300 may include a graphical representation of any of the components of the surgical robotic system. The graphical representation of patient platform 1306, the base 1313, and other components, may be rendered in real-time based on data received at the console 240, or may be loaded from a library, which may store rendered objects corresponding to various objects or humans in the operating room.

The stadium view model 1300 may also include a graphical representation of a patient 1315. In some embodiments, for example, when a patient is actually positioned on the patient platform, the graphical representation of the patient 1315 may be a rendering representing the actual physical size, shape, and anatomy of the patient. The rendering may be generated based on data received from various sensors positioned on the patient platform. For example, the graphical representation of the patient 1315 and the graphical representation of the patient platform 1306 may depict the actual height and width of the patient relative to the height and width of the patient platform. In other embodiments, the graphical representation of the patient 1315 may be a default render of a general human body anatomy displayed as being positioned on the graphical representation of the patient platform 1306. This default render of the human body may be stored in a library, which may be pre-loaded with rendered objects corresponding to various objects or humans.

In an embodiment, the stadium view model 1300 may not depict any instrument 212A, 212B information. Instead, the stadium view model 1300 may be focused on the positioning and status of various elements of the surgical robotic system 203, the patient, and other staff/objects in the operating room.

Each of the view icons 1310A-1310D may correspond to a different view of the stadium view model 1300. For example, icon 1310A may correspond to a second view of the stadium view model 1300, which may be a birds-eye view of the environment around the surgical robotic system 203. The second view may display the components of the surgical robotic system 203 (e.g., the graphical representation of the robotic arms 1303A-1303D, patient platform 1306, and portions of the base 1313) from a top-down perspective. The second view may also provide a graphical representation of the patient 1315 from a top-down perspective. In an embodiment, the medical provider may select the view icon 1310A by providing a user input to a user input device of the console 240 (e.g., to a touchscreen interface of the touchscreen 232) to cause the second view of the stadium view model 1300 to be displayed at the console 240. After selection of the view icon 1310A, the view icon 1310A may be highlighted with a different background color (e.g., blue).

For example, icon 1310C may correspond to a third view of the stadium view model 1300, which may be from a lower perspective (e.g., from the perspective near the foot of the patient platform 1306 or patient 1315). The third view may similarly display the components of the surgical robotic system 203 (e.g., the graphical representation of the robotic arms 1303A-1303D, patient platform 1306, and portions of the base 1313), but from the lower perspective. The third view may also provide a graphical representation of the patient 1315 from the lower perspective. In an embodiment, the medical provider may select the view icon 1310C by providing a user input to a user input device of the console 240 (e.g., to a touchscreen interface of the touchscreen 232) to cause the third view of the stadium view model 1300 to be displayed at the console 240. After selection of the view icon 1310C, the view icon 1310C may be highlighted with a different background color (e.g., blue).

For example, icon 1310D may correspond to a fourth view of the stadium view model 1300, which may be from a higher perspective (e.g., similar to the view shown in FIG. 3, but from a different side of the patient). The fourth view may similarly display the components of the surgical robotic system 203 (e.g., the graphical representation of the robotic arms 1303A-D, patient platform 1306, and portions of the base 1313), but from the higher perspective. The fourth view may also provide a graphical representation of the patient 1315 from the higher perspective. In an embodiment, the medical provider may select the view icon 1310D by providing a user input to a user input device of the console 240 (e.g., to a touchscreen interface of the touchscreen 232) to cause the fourth view of the stadium view model 1300 to be displayed at the console 240. After selection of the view icon 1310D, the view icon 1310D may be highlighted with a different background color (e.g., blue).

Therefore, the stadium view model 1300 includes multiple pre-defined views or perspectives of the environment around the surgical robotic system 203, such that a medical provider may access and switch between the pre-defined views using the view icons 1310A-1310D as needed during a procedure without removing from immersion. While only four views of the stadium view model 1300 are discussed herein, it should be appreciated that the stadium view model 1300 may include any number of views depicting the surgical robotic system 203 (or any robotic system) from different perspectives.

In another embodiment, a single stadium view model 1300 may be presented, in which the stadium view model 1300 is displayed with, for example, a slider user interface element. The slider user interface element may be dragged left and right and/or up and down, which may correspondingly rotate the stadium view model 1300 left, right, up, and down to present different views of the stadium view model 1300. As mentioned above, the stadium view model 1300, regardless of the view being displayed, may accurately depict a current position and orientation of different objects and humans in the operating room, including the patient, each of the robotic arms 210, the patient platform, etc.

### F. Destination Screen Model

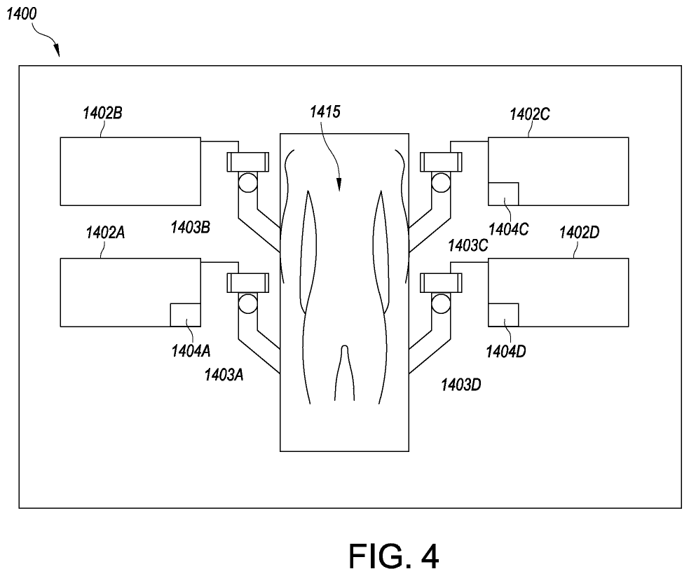

FIG. 4 illustrates a destination screen model 1400 displayed at the console 240 of a surgical robotic system 203 according to various embodiments of the disclosure. However, it should be appreciated that the destination screen model 1400 may be generated for other types of robotic systems throughout various different industries. In an embodiment, the destination screen model 1400 may include all of the components of the instrument-to-arm mapping model 1200, but may also include a graphical representation of the patient 1415. In the embodiment shown in FIG. 4, the destination screen model 1400 may not depict the entire patient, but instead may only depict graphical representations of portions of the patient and the surgical robotic system that are relevant to the medical provider (e.g., only a portion of the patient at which the robotic arms 210 may be performing a procedure). In other embodiments, the destination screen model 1400 may depict the entire patient 1415.

The destination screen model 1400 may include a graphical representation of the robotic arms 1403A-1403D, which may represent the actual position of the robotic arms 210 at the table-side. The destination screen model 1400 may, in some embodiments, depict the distal end of the robotic arms 1403A-1403D, which may comprise the distal links and joints that are unlocked and capable of moving.

The destination screen model 1400 may also include instrument indicators 1402A-1402D (similar to the instrument indicators 1203A-1203D), describing instruments 212A, 212B coupled to the robotic arms 210 represented by the robotic arms 1403A-1403D. The destination screen model 1400 may also include the HID indicators 1404A, 1404C, 1404D (similar to the HID indicators 1215A-1215B, 1215D), describing the handedness or hand assignments between the robotic arms 1403A-1403D/instruments 212A, 212B described by the instrument indicators 1402A-1402D and the HIDs 226, 228.

The destination screen model 1400 may be a default screen that displays at a display device 242 or the touchscreen 232 of the console 240. For example, the destination screen model 1400 may be set to display at the touchscreen 232 by default at all times during set-up and performing of a procedure, unless the medical provider interacts with the touchscreen 232 to change the display to a different screen or window (e.g., to display the instrument-to-arm mapping model 1200 of FIGS. 2A-2D or the stadium view model 1300 of FIG. 3).

### G. Status Model

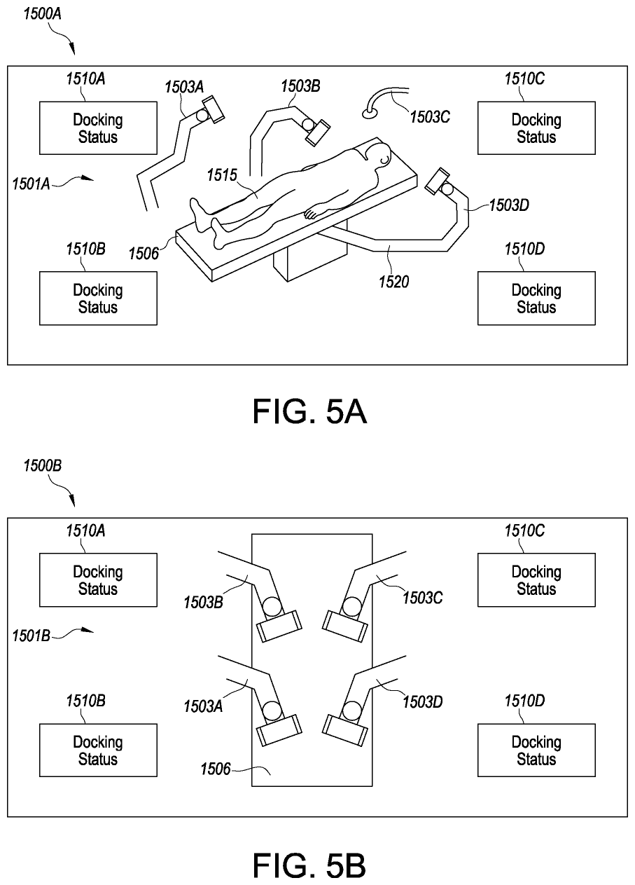

Turning now to FIGS. 5A-B, shown are various examples of a status models 1500A-1500B displayed at a console 240 of a surgical robotic system 203 according to various embodiments of the disclosure. However, it should be appreciated that the status models 1500A-1500B may be generated for other types of robotic systems throughout various different industries. The status models 1500A-1500B may indicate a current position, orientation, and status of one or more joints and/or links along each robotic arm 210 deployed at a table-side of a surgical robotic system 203. In one embodiment, the status models 1500A-1500B may be represented in zoomed-out fashion in which the entirety of the patient, robotic arms 210, patient platform, and base may be represented, similar to the stadium view model 1300. In another embodiment, the status models 1500A-1500B may be represented in a zoomed-in fashion in which only the unlocked portion of the robotic arms 210 are depicted and emphasized.

Referring now to FIG. 5A, shown is a first view 1501A depicting a status model 1500A and docking status indicators 1510A-D according to various embodiments of the disclosure. The status model 1500A includes a graphical rendering of the robotic arms 1503A-1503D, reflecting a current position and orientation of the robotic arms 210 relative to a table-side. The status model 1500A also includes a graphical representation of the patient platform 1506, representing the patient platform at the table-side. The status model 1500A also includes a graphical representation of the patient 1515, in which the entirety of the patient is depicted (though in some embodiments, the entirety of the patient 1515 need not be depicted). The status model 1500A may include graphical representations of other humans or objects in the operating room that are not rendered in the status model 1500A shown in FIG. 5A. The graphical representation of robotic arms 1503A-1503D, patient platform 1506, patient 1515, and other objects/humans may be rendered in real-time using a rendering application or may be obtained from a library that may be loaded with these graphical representations.

In an embodiment, the graphical representation of the robotic arms 1503A-1503D may also include graphical representations for each joint and/or link along the robotic arms 1503A-1503D. Referring to the graphical representation of the robotic arm 1503D specifically, the robotic arm 1503D includes graphical representations of multiple links 1520. The graphical representations of the links 1520 may each be rendered to represent the physical structure of each of the links at the table-side.

In some embodiments, the positions and orientations of each of the robotic arms 1503A-1503D in the status model 1500A may reflect a current position and/or orientation of each of the corresponding robotic arms 210 at the table-side. Similarly, the positions and/or orientations of each of the links 1520 within the robotic arms 1503A-1503D may reflect a current position and/or orientation of each of the links 1520 along the robotic arms 210 at the table-side.

In an embodiment, the status model 1500A may reflect the status of each of the links along the robotic arms 210 by varying a visual factor of each of the graphical representations of the links 1520 along each of the robotic arms 1503A-1503D based on the status of the link. The status of each of the links 1520 may refer to whether a particular link on a robotic arm 210 is unlocked and permitted to move or locked and prohibited from moving. When a link is unlocked and permitted to move, the position and the orientation of the link may be significant to the medical provider operating the robotic arms 210. This may be because movement of the robotic arms 210 may adversely affect set-up of the system or performance of the procedure. Meanwhile, when a link is locked and prohibited from moving, the position and the orientation of the link may not be as significant to the medical provider operating the robotic arms 210. This may be because the locked robotic arms 210 may have little to no effect on the set-up of the system or the performance of the procedure. For this reason, providing a clear indication of a status of the links 1520 along the robotic arms 210 that are deployed may be significantly helpful to the medical provider.

The status model 1500A illustrates a pre-procedure position of the robotic arms 210. In the pre-procedure position, the robotic arms 210 are deployed above the patient platform but relatively distant from the patient platform 1506 (e.g., not positioned above a patient anatomy for a particular procedure). In the pre-procedure position, all of the links and joints along the robotic arms 210 may be unlocked and moved, since the robotic arms 210 are in the process of being set up for the procedure at this stage.

The status of each of the links along the robotic arms 210 may be indicated in the status model 1500A by varying a visual factor of each of the graphical representations of the links 1520 along each of the robotic arms 1503A-1503D. For example, the graphical representations of each of the links 1520 may be set to a first color when the status of the corresponding link at the table-side is unlocked and movable, and set to a second color when the status of the corresponding link at the table-side is locked and otherwise prohibited from moving. As another example, graphical representations of each of the links 1520 may be set to be a solid color when the status of the corresponding link at the table-side is unlocked and movable, and set to a shaded or greyed-out color when the status of the corresponding link at the table-side is locked and otherwise prohibited from moving. In either case, the visual factor indicating that the link is movable may be brighter or easier to see than the visual factor indicating the link is immovable, thereby highlighting the relevant movable links that may affect set-up or performance of the procedure, while dimming out the links that may not be relevant to the medical provider during set-up or performance of the procedure. In this way, the status model 1500A selectively highlights certain aspects of the surgical robotic system 203 for the medical provider to focus on while dimming out other irrelevant aspects of the surgical robotic system 203 that would otherwise distract the medical provider during set-up and performance of the procedure.

In addition, the status model 1500A may additionally include the docking status indicators 1510A-1510D. The docking status indicators 1510A-1510D may convey the status of an entire robotic arm 210 at the table-side. A robotic arm 210 may be docked, for example, into a pre-defined position when one or more pre-defined links on the robotic arm 210 are locked into position. The docking status indicator 1510A-1510D may be represented as associated with a robotic arm 210 in a variety of different manners. For example, the graphical representation of the robotic arm 1503A-1503D may be positioned most proximate to the graphical representation of the robotic arm 1503A-1503D. For example, the docking status indicators 1510A-1510D may be positioned from left-to-right or right-to-left to correspond with the graphical representations of the robotic arms 1503A-1503D positioned in a particular order from left-to-right or right-to-left. As shown in FIG. 5A, the robotic arm 1503A is most proximate to the docking status indicator 1510B, and thus the docking status indicator 1510B describes whether the table-side robotic arm 210 represented by the robotic arm 1503A is docked, for example, into a pre-defined position. Similarly, the robotic arm 1503B is most proximate to the docking status indicator 1510A, and thus the docking status indicator 1510A describes whether the table-side robotic arm 210 represented by the robotic arm 1503B is docked. The robotic arm 1503C is most proximate to the docking status indicator 1510C, and thus the docking status indicator 1510C describes whether the table-side robotic arm 210 represented by the robotic arm 1503C is docked. Lastly, the robotic arm 1503D is most proximate to the docking status indicator 1510D, and thus the docking status indicator 1510D describes whether the table-side robotic arm 210 represented by the robotic arm 1503D is docked.

The docking status indicators 1510A-1510D may include an identifier of the corresponding robotic arm 210. The docking status indicators 1510A-1510D may also include text indicating whether the corresponding table-side robotic arm 210 is docked. For example, the text may recite either “Docked” to indicate the robotic arm 210 is docked, or “Undocked” to indicate the robotic arm 210 is not docked.

Referring now to FIG. 5B, shown is a second view 1501B depicting a status model 1500B and docking status indicators 1510A-1510D according to various embodiments of the disclosure. Unlike the first view 1501A of the status model 1500A shown in FIG. 5A, the second view 1501B of the status model 1500B shown in FIG. 5B is zoomed in, and does not include a graphical representation of a patient. Moreover, the position of the robotic arms 1503A-1503D are different (e.g., a procedure position). Such a position of the corresponding physical robotic arms 210 may be used in, for example, an upper abdominal procedure performed on a patient.

In this case, the links 1520 on the robotic arms 1503A-1503D may be set to remain in a first color (e.g., white), which may be a highlighted color indicating that the links on the corresponding table-side robotic arms 210 are unlocked and movable. Similarly, the docking status indicators 1510A-1510D may also indicate that the table-side robotic arms 210 corresponding to the robotic arms 1503A-1503D are all still undocked.

In some embodiments, the status models 1500A-1500B may be updated to reflect that some of the robotic arms 210 are docked while some of the robotic arms 210 are still undocked. For example, the status models 1500A-1500B may be updated to reflect that the table-side robotic arms 210 corresponding to the graphical representations of the robotic arms 1503A, 1503C, 1503D are docked, while the table-side robotic arm 210 corresponding to the graphical representation of the robotic arm 1503B is undocked. This change in status may be reflected in the status models 1500A-1500B in a few different ways. For example, the docking status indicators 1510A, 1510C, 1510D may be updated to include the text “Docked” to indicate that the robotic arms 210 corresponding to the respective docking status indicators 1510A, 1510C, 1510D are docked. In addition, the visual factor of some of the links 1520 on the robotic arms 1503A, 1503C, 1503D may be adjusted in color or shading to be dulled out (e.g., changed to grey, black, or shaded). This change to the visual factor of the links 1520 may indicate that the corresponding table-side links 1520 of the robotic arm 210 have been locked and are immovable. Meanwhile, some of the other links 1520 on the robotic arm 1503A may remain highlighted in a brighter color (e.g., white) in the status model 1500A-1500B. The brighter color of links may indicate that the corresponding table-side links of the robotic arm 210 are unlocked and permitted to move (therefore, significant to the medical provider in terms of focus).

In some embodiments, the status models 1500A-1500B may be updated to reflect that some of the robotic arms 210 have been coupled to an instrument 212, while others may still not be coupled to an instrument 212. For example, the status models 1500A-1500B may be updated to reflect that three of the table-side robotic arms 210 corresponding to the graphical representations of the robotic arms 1503B, 1503C, 1503D have been coupled to an instrument 212A, 212B while the table-side robotic arm 210 corresponding to the graphical representation of the robotic arm 1503A is docked. These changes in status and coupled instruments 212A, 212B may be reflected in the status models 1500A-1500B in a few different ways. For example, the docking status indicators 1510B, 1510C, 1510D may have been updated to include instrument indicators, such as the instrument indicators 1203A-1203D described above with reference to FIGS. 2A-2D, when an instrument 212A, 212B is coupled to the respective robotic arm 210. The instrument indicators may include icons, images (e.g., camera images or pre-stored digital rendering models), text, identifiers, and/or other data used to describe and identify a particular instrument 212A, 212B coupled to the corresponding table-side robotic arm 210. Meanwhile, the docking status indicator 1510A of the robotic arm 1503A may be updated to include the text “Docked” to indicate that the corresponding table-side robotic arm 210 is docked (however, an instrument 212A, 212B may not yet be coupled to that corresponding table-side robotic arm 210).

### H. Display Device and Touchscreen at the Console

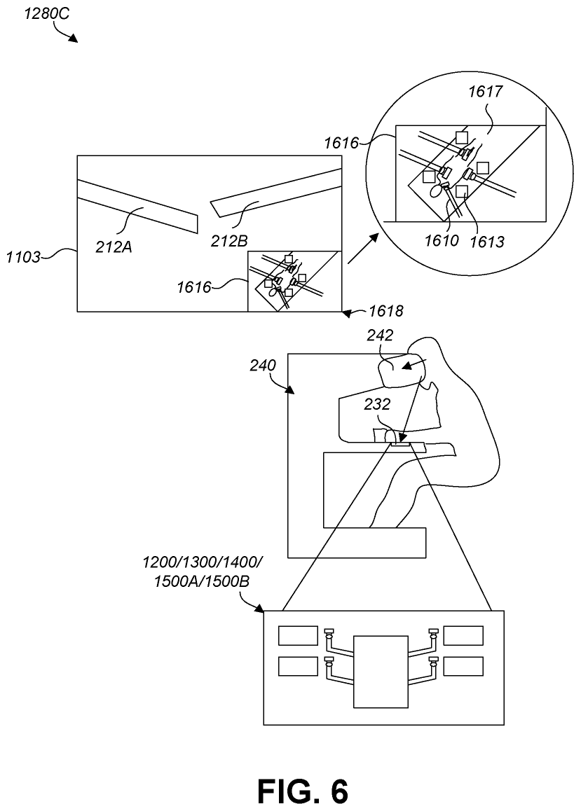

FIG. 6 is a diagram 1600 illustrating a manner of displaying the instrument-to-arm mapping model 1200, the stadium view model 1300, the destination screen model 1400, and the status models 1500A, 1500B, or a modified version of the models 1200, 1300, 1400, 1500A, and/or 1500B according to various embodiments of the disclosure. As mentioned above, the console 240 may include multiple displays. For example, one display may be located in the display device 242, which may be embodied as a headset. Another display may be located at the touchscreen 232, which may be positioned on a handle of the console 240, for example.

In an embodiment, one or more of the instrument-to-arm mapping model 1200, the stadium view model 1300, the destination screen model 1400, and the status models 1500A, 1500B may be displayed at the display device 242. As shown in FIG. 6, one or more of the models 1200, 1300, 1400, 1500A, 1500B disclosed herein may be displayed at the display device 242 in a picture-in-picture (PIP) format, in which the models 1200, 1300, 1400, 1500A, 1500B is positioned in a small box 1616 positioned at the corner 1618 of the display or the image 1103. In this case, the models 1200, 1300, 1400, 1500A, 1500B may be rendered and overlaid on top of the image 1103. While FIG. 6 shows the corner 1618 positioned at the bottom right of the image 1103, the models 1200, 1300, 1400, 1500A, 1500B may be displayed in a PIP format at any position or corner of the image 1103.

In an embodiment, a modified model 1617 may be displayed at the display device 242 in the PIP format. The modified model 1617 may a simpler or less detailed version of the instrument-to-arm mapping model 1200, the stadium view model 1300, the destination screen model 1400, and/or the status models 1500A, 1500B. The modified model 1617 displayed in the PIP format may include less data (e.g., text, icons, images, etc.) compared to the other models 1200, 1300, 1400, 1500A, and/or 1500B. In the example shown in FIG. 6, the modified model 1617 may include graphical representations 1610 of the robotic arms 210 and/or the instruments 212A-B. The modified model 1617 may include HID indicators 1613 depicting information regarding an instrument 212 coupled to a robotic arm 210. Each HID indicator 1613 may be proximate to a graphical representation 610 of a robotic arm 210, and thus may represent information regarding an instrument 212 coupled to the robotic arm 210 represented by the proximate graphical representation 610. The HID indicators 1613 may indicate a handedness, engagement status, and/or other data related to an instrument 212. The HID indicators 1613 may each include an icon, image, or text identifying an instrument 212 coupled to a robotic arm 210. For example, a HID indicator 1613 may include an icon of a camera when the instrument 212 being represented is a scope device or camera. The HID indicators 1613 may include an icon of a left hand or an icon of a right hand, depending on whether the instrument 212 is being actively controlled by the left HID 226 or the right HID 228. The HID indicators 1613 may include the text “L” or “H” with a circular arrow around the text when an instrument 212 disengaged from an HID 226, 228, but is available for swapping by a respective left HID 226 or a right HID 228. An outline of the HID indicators 1613 may also indicate, based on color or brightness for example, whether the instrument 212 is actively engaged by a HID 226, 228 or is disengaged from the HID 226, 228. In some cases, the modified model 1617 may also include a graphical representation of the patient, such that the modified model 1617 depicts a position of each of the instruments 212 and/or robotic arms 210 relative to a position of the patient.

In some embodiments, the modified model 1617 may depict a particular perspective view or a cropped area of the stadium view model 1300 based on the components of the surgical robotic system that are most relevant to the medical provider during set-up or performing of the procedure. For example, if only one robotic arm 210 is active with an instrument 212 and another robotic arm 210 actively operates a camera, the stadium view model 1300 may only display the position and orientation of the two robotic arms 210 operating the camera and the sole instrument (e.g., the stadium view model 1300 may be cropped to exclude any renderings of inactive robotic arms 210, that would unnecessarily consume space at the display device 242).

When the models 1200, 1300, 1400, 1500A, 1500B, 1617 are displayed in the PIP format, the models 1200, 1300, 1400, 1500A, 1500B, 1617 may be selectively displayed at the display device 242 in response to a user input received at a user input device of the console 240. For example, the console 240 may include multiple different user input devices (e.g., buttons, switches, touch-sensitive surfaces, gimbals, toggles, pedals, etc.) positioned, for example, on the HIDs 226, 228, on an armrest, and/or at a foot actuator assembly of the console 240. The medical provider may provide the user input or a combination of user inputs across one or more of the input devices at the console 240 to trigger display of the models 1200, 1300, 1400, 1500A, 1500B, 1617 in the PIP format at the display device 242.

For example, the medical provider may desire to view the stadium view model 1300 or the modified model 1617 when access to the position of the various robotic arms 210 with reference to the patient platform is desired, but the medical provider does not want to remove from immersion. In this case, the medical provider may provide one or more user inputs to the console 240, which may trigger the PIP display of the stadium view model 1300 or the modified model 1617 at the display device 242. In some cases, the models 1200, 1300, 1400, 1500, 1500A, 1500B, 1617 may only be displayed at the display device 242 in the PIP format when the user input is provided to the console 240 (i.e., the medical provider may need to constantly press the foot pedal at the foot actuator assembly and/or push a button at the HID 226, 228 for the model 1200, 1300, 1400, 1500A, 1500B, 1617 to be displayed in the PIP format). In this case, the model 1200, 1300, 1400, 1500A, 1500B, 1617 may discontinue being displayed at the display device 242 when the medical provider stops providing the user input.

Similarly, the medical provider may desire to view the instrument-to-arm mapping model 1200 when knowledge of the instruments 212 coupled to one or more of the robotic arms 210 are desired, but the medical provider does not want to remove from immersion. In this case, the medical provider may provide one or more user inputs to the console 240, which may trigger the PIP display of the instrument-to-arm mapping model 1200 at the display device 242.

In an embodiment, the destination screen model 1400 may be a default screen that displays at the touchscreen 232 of the console 240. For example, the destination screen model 1400 may be set to display by default at the touchscreen 232 at all times during set-up and performing of a procedure, unless the medical provider interacts with the touchscreen 232 (e.g., selects an icon on the touchscreen 232 interface) to display a particular model 1200, 1300, or 1500A, 1500B, 1617.

For example, during set-up of the procedure, before the medical provider begins performing incisions or examinations robotically on the patient, the medical provider may need to set up the initial positions of the robotic arms 210 and instruct staff to couple specific instruments 212 or cameras to one or more of the robotic arms 210. Therefore, the medical provider may view, for example, the status model 1500A, 1500B displayed at the touchscreen 232 to facilitate set-up of the positioning and locking/unlocking of each of the robotic arms 210 prior to immersing into the display device 242 and beginning the procedure on the patient.

As another example, the medical provider may be performing the procedure on the patient and immersed into the display device 242 while noticing that one of the instruments 212 has limited movement, and this limited movement may be shown in the image 1103 displayed at the display device 242. However, a cause of the limited movement may be unlikely to be shown at the image 1103 because very limited information may be displayed at the display device 242. To fix this issue, the medical provider may temporarily remove from immersion (which locks the robotic arms 210 in some embodiments for safety purposes) and view the touchscreen 232 to diagnose a cause of this limited movement. As mentioned above, the touchscreen 232 may be, by default, set to display the destination screen model 1400. In some cases, the destination screen model 1400 may indicate that two robotic arms 210 are relatively close together and may be colliding. The medical provider may however provide one or more user inputs, using one or more user input devices, at the console 240 to display a stadium view model 1300 or modified model 1617, which may provide a zoomed-out view of the position and orientation of all of the deployed robotic arms 210. The medical provider may use the zoomed-out stadium view model 1300, modified model 1617, and/or the destination screen model 1400 to troubleshoot a cause of this limited movement, and may act accordingly (i.e., robotically or manually correct the positioning of the robotic arms 210, or request staff closer to the table-side to correct the positioning of the robotic arms 210).

The robotic system may in some cases include a tower, which may be separate from the console 240 and the robotic arms 210. The tower may provide support for controls, electronics, fluidics, optics, sensors, and/or power for robotic arms 210 and/or the console 240. In some embodiments, the tower includes a display device. The display device at the tower may display one or more of the instrument-to-arm mapping model 1200, the stadium view model 1300, the destination screen model 1400, the status models 1500A, 1500B, and/or the modified model 1617. The different models 1200, 1300, 1400, 1500A, 1500B, and/or 1617 may be displayed periodically throughout the procedure, for example, based on user input at the console or based on a stage of the procedure.

### I. Exemplary Processes.

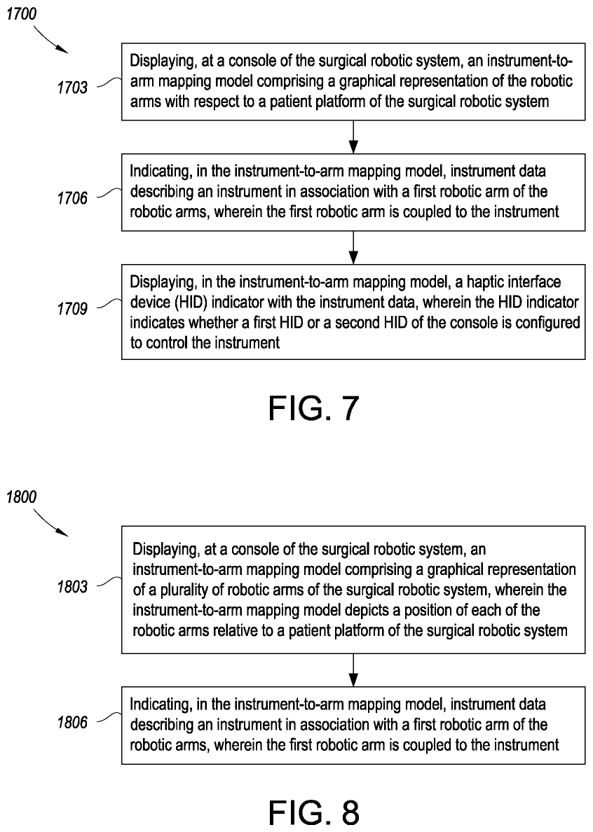

FIG. 7 is a flowchart illustrating method 1700 performed by a robotic system. Specifically, method 1700 may be performed by a processor in the console 240 or coupled to the console 240.

At step 1703, method 1700 comprises displaying, at a console 240 of the surgical robotic system, an instrument-to-arm mapping model 1200 comprising a graphical representation of the robotic arms 210 with respect to a patient platform of the surgical robotic system 203. The graphical representation of the robotic arms may be rendered as the robotic arms 1230A-1230D of FIG. 2A, the robotic arms 1303A-1303D of FIG. 3, the robotic arms 1403A-1403D of FIG. 4, or the robotic arms 1503A-1503D of FIGS. 5A-5B.

At step 1706, method 1700 comprises indicating, in the instrument-to-arm mapping model 1200, instrument data describing an instrument 212A, 212B in association with a first robotic arm 210 of the robotic arms 210, wherein the first robotic arm 210 is coupled to the instrument 212A, 212B. The instrument data may be displayed in the instrument-to-arm mapping model 1200 as the instrument indicators 1203A-1203D shown in FIG. 2A.

At step 1709, method 1700 comprises displaying, in the instrument-to-arm mapping model 1200, HID indicators 1215A-1215B, 1215D with the instrument data. The HID indicator 1215A-1215B, 1215D indicates whether a first HID 226 or a second HID 228 of the console 240 is configured to control the instrument 212A, 212B coupled to the first robotic arm 210.

FIG. 8 is a flowchart illustrating method 1800 performed by the surgical robotic system 200 or 400. Specifically, method 1800 may be performed by a processor in the console 240 or coupled to the console 240.

At step 1803, method 1800 comprises displaying, at a console 240 of the surgical robotic system, an instrument-to-arm mapping model 1200 comprising a graphical representation of a plurality of robotic arms 210 of the surgical robotic system 203. The instrument-to-arm mapping model 1200 depicts a position of each of the robotic arms 210 relative to a patient platform 1234 of the surgical robotic system 203. The graphical representation of the robotic arms may be rendered as the robotic arms 1230A-1230D of FIG. 2A, the robotic arms 1303A-1303D of FIG. 3, the robotic arms 1403A-1403D of FIG. 4, or the robotic arms 1503A-1503D of FIGS. 5A-5B.

At step 1806, method 1800 comprises indicating, in the instrument-to-arm mapping model 1200, instrument data describing an instrument 212A, 212B in association with a first robotic arm 210 of the robotic arms 210, wherein the first robotic arm 210 is coupled to the instrument 212A, 212B. The instrument data may be displayed in the instrument-to-arm mapping model 1200 as the instrument indicators 1203A-1203D shown in 2A.

### J. Implementing Systems and Terminology.

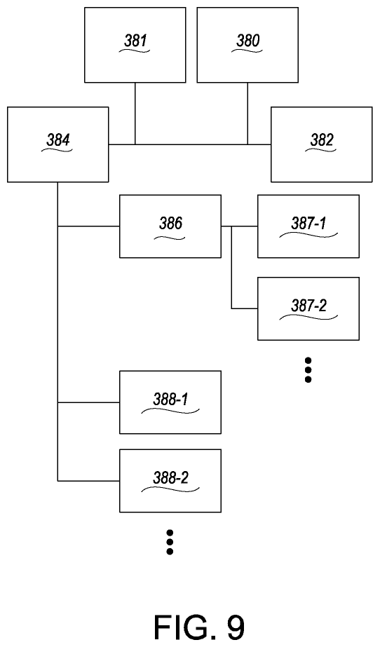

FIG. 9 is a schematic diagram illustrating electronic components of a surgical robotic system in accordance with some embodiments.

The surgical robotic system, such as surgical robotic system 203, includes one or more processors 380, which are in communication with a computer-readable storage medium 382 (e.g., computer memory devices, such as random-access memory, read-only memory, static random-access memory, and non-volatile memory, and other storage devices, such as a hard drive, an optical disk, a magnetic tape recording, or any combination thereof) storing instructions for performing any methods described herein (e.g., operations described with respect to FIGS. 1-9). The one or more processors 380 are also in communication with an input/output controller 384 (via a system bus or any suitable electrical circuit). The input/output controller 384 receives sensor data from one or more sensors 388-1, 388-2, etc., and relays the sensor data to the one or more processors 380. The input/output controller 384 also receives instructions and/or data from the one or more processors 380 and relays the instructions and/or data to one or more actuators, such as first motors 387-1 and 387-2, etc. In some embodiments, the input/output controller 384 is coupled to one or more actuator controllers 386 and provides instructions and/or data to at least a subset of the one or more actuator controllers 386, which, in turn, provide control signals to selected actuators. In some embodiments, the one or more actuator controllers 386 are integrated with the input/output controller 384 and the input/output controller 384 provides control signals directly to the one or more motors 387-1 and 387-2, etc. (without a separate actuator controller). Although FIG. 9 shows that there is one actuator controller 386 (e.g., one actuator controller for the entire surgical robotic system; in some embodiments, additional actuator controllers may be used (e.g., one actuator controller for each actuator, etc.). In some embodiments, the one or more processors 380 are in communication with one or more displays 381 for displaying information as described herein.

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.
Example Combination 1: A surgical robotic system, may include: a plurality of robotic arms may include a first robotic arm, where the first robotic arm is coupled to an instrument; and a console communicatively coupled to the robotic arms, may include: a display device; and a processor coupled to the display device and configured to: display, at the display device, an instrument-to-arm mapping model may include a graphical representation of the robotic arms, where the instrument-to-arm mapping model depicts a position of each of the robotic arms; and indicate, in the instrument-to-arm mapping model, instrument data describing the instrument in association with the first robotic arm.
Example Combination 2: The surgical robotic system of Example Combination 1, where, to indicate the instrument data in association with the first robotic arm, the display is further configured to display a call line coupling the instrument data to the first robotic arm.
Example Combination 3: The surgical robotic system of any of Example Combination 1 or Example Combination 2, where the graphical representation of the robotic arms may include a rendering of each of the robotic arms.
Example Combination 4: The surgical robotic system of any of any one of Example Combinations 1-3, where the rendering of each of the robotic arms includes a rendering of one or more joints and links of a robotic arm.
Example Combination 5: The surgical robotic system of any of any one of Example Combinations 1-4, where the instrument data may include at least one of an identifier of the first robotic arm, an image depicting the instrument, text describing the instrument, an image indicating an orientation of the instrument, or text describing an operation performable by the instrument.
Example Combination 6: The surgical robotic system of any of any one of Example Combinations 1-5, where the instrument-to-arm mapping model further may include a haptic interface device (HID) indicator displayed with the instrument data, where the HID indicator indicates a HID of the console engaged to control the instrument.
Example Combination 7: The surgical robotic system of any of any one of Example Combinations 1-6, where the instrument-to-arm mapping model further may include a haptic interface device (HID) indicator displayed with the instrument data, where the HID indicator indicates whether a HID of the console is configured to control the instrument but not currently engaged to control the instrument.
Example Combination 8: The surgical robotic system of any of any one of Example Combinations 1-7, where the robotic arms further may include a second robotic arm, where the second robotic arm is coupled to a camera, where the instrument data may include scope data, where the scope data may include at least one of an angle of the camera or a direction of the camera.
Example Combination 9: The surgical robotic system of any of any one of Example Combinations 1-8, where the processor is further configured to display, at the display device, an image of a patient, where the instrument-to-arm mapping model is overlaid over a portion of the image of the patient.
Example Combination 10: The surgical robotic system of any of any one of Example Combinations 1-9, where the instrument data is indicated in an icon displayed at the display device.
Example Combination 11: The surgical robotic system of any of any one of Example Combinations 1-10, where the display device is positioned in a headset of the console or on an armrest of the console.
Example Combination 12: A method performed by a surgical robotic system, may include: displaying, at a console of the surgical robotic system, an instrument-to-arm mapping model may include a graphical representation of a plurality of robotic arms of the surgical robotic system, where the instrument-to-arm mapping model depicts a position of each of the robotic arms relative to a patient platform of the surgical robotic system; and indicating, in the instrument-to-arm mapping model, instrument data describing an instrument in association with a first robotic arm of the robotic arms, where the first robotic arm is coupled to the instrument.
Example Combination 13: The method of Example Combination 12, where indicating the instrument data in association with the first robotic arm may include displaying a call line coupling the instrument data to the first robotic arm.
Example Combination 14: The method of any of Example Combination 12 or Example Combination 13, where the graphical representation of the robotic arms may include a rendering of each of the robotic arms, and where the method further may include updating the position of the rendering of each of the robotic arms based on an actual position of the robotic arms.
Example Combination 15: The method of any of any one of Example Combinations 12-14, where the instrument data may include at least one of an identifier of the first robotic arm, an image depicting the instrument, text describing the instrument, an image indicating an orientation of the instrument, or text describing an operation performable by the instrument, where the instrument-to-arm mapping model further may include a haptic interface device (HID) indicator displayed with the instrument data, and where the HID indicator indicates an HID of the console engaged to control the instrument.
Example Combination 16: The method of any of any one of Example Combinations 12-15, further may include: receiving, by a processor of the console, one or more user inputs received at one or more user input devices of the console, and displaying, at the console, the instrument-to-arm mapping model in response to receiving the one or more user inputs.
Example Combination 17: The method of any of any one of Example Combinations 12-16, where the graphical representation of the robotic arms depicts at least one of a position, an angle, or orientation of one or more joints and links of each of the robotic arms.
Example Combination 18: The method of any of any one of Example Combinations 12-17, where the instrument data is indicated in an icon displayed at the console, where the method further may include: receiving, by a processor of the console, a selection of the icon; and displaying, at the console, a setting menu to adjust one or more settings of the instrument in response to receiving the selection of the icon.
Example Combination 19: The method of any of any one of Example Combinations 12-18, where the instrument-to-arm mapping model is displayed at a display device of the console, where the display device is positioned in a headset of the console or on an armrest of the console.
Example Combination 20: A non-transitory, computer-readable medium storing instructions which, when executed by a processor of a surgical robotic system may include a plurality of robotic arms, cause the processor to: display, at a console of the surgical robotic system, an instrument-to-arm mapping model may include a graphical representation of the robotic arms with respect to a patient platform of the surgical robotic system; indicate, in the instrument-to-arm mapping model, instrument data describing an instrument in association with a first robotic arm of the robotic arms, where the first robotic arm is coupled to the instrument; and display, in the instrument-to-arm mapping model, a haptic interface device (HID) indicator with the instrument data, where the HID indicator indicates whether a first HID or a second HID of the console is configured to control the instrument.
Example Combination 21: The non-transitory, computer-readable medium of Example Combination 20, where, to indicate the instrument data in association with the first robotic arm, the display is further configured to display a call line coupling the instrument data to the first robotic arm.
Example Combination 22: The non-transitory, computer-readable medium of any of Example Combination 20 or Example Combination 21, where the graphical representation of the robotic arms may include a rendering of each of the robotic arms, where the rendering of each of the robotic arms includes a rendering of one or more joints and links of a robotic arm.
Example Combination 23: The non-transitory, computer-readable medium of any of any one of Example Combinations 20-22, where the instrument data may include at least one of an identifier of the first robotic arm, an image depicting the instrument, text describing the instrument, an image indicating an orientation of the instrument, or text describing an operation performable by the instrument, where the instrument-to-arm mapping model further may include a haptic interface device (HID) indicator displayed with the instrument data, and where the HID indicator indicates an HID of the console configured to control the instrument.
Example Combination 24: The non-transitory, computer-readable medium of any of any one of Example Combinations 20-23, where the processor is further configured to display, at the console, an image of a patient, where the instrument-to-arm mapping model is overlaid over a portion of the image of the patient.
Example Combination 25: The non-transitory, computer-readable medium of any of any one of Example Combinations 20-24, where the console may include a display device, where the display device is positioned in a headset of the console or on an armrest of the console.
Example Combination 26: A surgical robotic system, may include: a patient platform; a plurality of robotic arms may include a first robotic arm, where the first robotic arm is coupled to an instrument; and a console communicatively coupled to the robotic arms, may include: a first haptic interface device (HID) and a second HID; and a display configured to: display an instrument-to-arm mapping model may include a graphical representation of the robotic arms with respect to a patient platform of the surgical robotic system; indicate, in the instrument-to-arm mapping model, instrument data describing an instrument in association with the first robotic arm; and display, in the instrument-to-arm mapping model, a haptic interface device (HID) indicator with the instrument data, where the HID indicator indicates whether a first HID or a second HID of the console is configured to control the instrument.
Example Combination 27: A method performed by a surgical robotic system may include a plurality of robotic arms, where the method may include: displaying, at a console of the surgical robotic system, an instrument-to-arm mapping model may include a graphical representation of the robotic arms with respect to a patient platform of the surgical robotic system; indicating, in the instrument-to-arm mapping model, instrument data describing an instrument in association with a first robotic arm of the robotic arms, where the first robotic arm is coupled to the instrument; and displaying, in the instrument-to-arm mapping model, a haptic interface device (HID) indicator with the instrument data, where the HID indicator indicates whether a first HID or a second HID of the console is configured to control the instrument.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The functions for determining whether a tool is within or outside a surgical field of view provided by a camera or scope and rendering one or more indicators representing positions or directions of one or more medical tools described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, the term “exemplary” means “serving as an example, instance, or illustration,” and does not necessarily indicate any preference or superiority of the example over any other configurations or implementations.

As used herein, the term “and/or” encompasses any combination of listed elements. For example, “A, B, and/or C” includes the following sets of elements: A only, B only, C only, A and B without C, A and C without B, B and C without A, and a combination of all three elements, A, B, and C.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the scope of the disclosure. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present disclosure is not intended to be limited to the embodiments shown herein but is to be accorded the widest scope consistent with the principles and novel features disclosed herein

1. A surgical robotic system, comprising:
a plurality of robotic arms comprising a first robotic arm, wherein the first robotic arm is coupled to an instrument; and
a console communicatively coupled to the robotic arms, comprising:
a display device; and
a processor coupled to the display device and configured to:
display, at the display device, an instrument-to-arm mapping model comprising a graphical representation of the robotic arms, wherein the instrument-to-arm mapping model depicts a position of each of the robotic arms; and
indicate, in the instrument-to-arm mapping model, instrument data describing the instrument in association with the first robotic arm.

2. The surgical robotic system of claim 1, wherein, to indicate the instrument data in association with the first robotic arm, the display is further configured to display a call line coupling the instrument data to the first robotic arm.

3. The surgical robotic system of claim 1, wherein the graphical representation of the robotic arms comprises a rendering of each of the robotic arms, wherein the processor is further configured to update a position of the rendering of each of the robotic arms based on an actual position of the robotic arms.

4. The surgical robotic system of claim 3, wherein the rendering of each of the robotic arms includes a rendering of at least one of a position, an angle, or orientation of one or more joints and links of a robotic arm.

5. The surgical robotic system of claim 1, wherein the instrument data comprises at least one of an identifier of the first robotic arm, an image depicting the instrument, text describing the instrument, an image indicating an orientation of the instrument, or text describing an operation performable by the instrument.

6. The surgical robotic system of claim 1, wherein the instrument-to-arm mapping model further comprises a haptic interface device (HID) indicator displayed with the instrument data, wherein the HID indicator indicates a HID of the console engaged to control the instrument.

7. The surgical robotic system of claim 1, wherein the instrument-to-arm mapping model further comprises a haptic interface device (HID) indicator displayed with the instrument data, wherein the HID indicator indicates whether a HID of the console is configured to control the instrument but not currently engaged to control the instrument.

8. The surgical robotic system of claim 1, wherein the robotic arms further comprise a second robotic arm, wherein the second robotic arm is coupled to a camera, wherein the instrument data comprises scope data, wherein the scope data comprises at least one of an angle of the camera or a direction of the camera.

9. The surgical robotic system of claim 1, wherein the processor is further configured to display, at the display device, an image of a patient, wherein the instrument-to-arm mapping model is overlaid over a portion of the image of the patient.

10. (canceled)

11. The surgical robotic system of claim 1, wherein the display device is positioned in a headset of the console or on an armrest of the console.

12. The surgical robotic system of claim 1, wherein the instrument-to-arm mapping model depicts a position of each of the robotic arms relative to a patient platform of the surgical robotic system.

13. The surgical robotic system of claim 1, wherein the processor is further configured to:
receive one or more user inputs received at one or more user input devices of the console, and
display, at the console, the instrument-to-arm mapping model in response to receiving the one or more user inputs.

14. The surgical robotic system of claim 1, wherein the instrument data is indicated in an icon displayed at the console, wherein the processor is further configured to:
receive a selection of the icon; and
display, at the console, a setting menu to adjust one or more settings of the instrument in response to receiving the selection of the icon.

15. The surgical robotic system of claim 1, wherein the processor is further configured to:
display, in the instrument-to-arm mapping model, a haptic interface device (HID) indicator with the instrument data, wherein the HID indicator indicates whether a first HID or a second HID of the console is configured to control the instrument.

16. A method performed by a surgical robotic system, comprising:
displaying, at a console of the surgical robotic system, an instrument-to-arm mapping model comprising a graphical representation of a plurality of robotic arms of the surgical robotic system, wherein the instrument-to-arm mapping model depicts a position of each of the robotic arms relative to a patient platform of the surgical robotic system; and
indicating, in the instrument-to-arm mapping model, instrument data describing an instrument in association with a first robotic arm of the robotic arms, wherein the first robotic arm is coupled to the instrument.

17. The method of claim 16, wherein indicating the instrument data in association with the first robotic arm comprises displaying a call line coupling the instrument data to the first robotic arm.

18. The method of claim 16, wherein the graphical representation of the robotic arms comprises a rendering of each of the robotic arms, and wherein the method further comprises updating the position of the rendering of each of the robotic arms based on an actual position of the robotic arms.

19. The method of claim 16, wherein the instrument data comprises at least one of an identifier of the first robotic arm, an image depicting the instrument, text describing the instrument, an image indicating an orientation of the instrument, or text describing an operation performable by the instrument, wherein the instrument-to-arm mapping model further comprises a haptic interface device (HID) indicator displayed with the instrument data, and wherein the HID indicator indicates an HID of the console engaged to control the instrument.

20. (canceled)

21. (canceled)

22. The method of claim 16, wherein the instrument data is indicated in an icon displayed at the console, wherein the method further comprises:
receiving, by a processor of the console, a selection of the icon; and
displaying, at the console, a setting menu to adjust one or more settings of the instrument in response to receiving the selection of the icon.

23. (canceled)

24. A non-transitory, computer-readable medium storing instructions which, when executed by a processor of a surgical robotic system comprising a plurality of robotic arms, cause the processor to:
display, at a console of the surgical robotic system, an instrument-to-arm mapping model comprising a graphical representation of the robotic arms with respect to a patient platform of the surgical robotic system;
indicate, in the instrument-to-arm mapping model, instrument data describing an instrument in association with a first robotic arm of the robotic arms, wherein the first robotic arm is coupled to the instrument; and
display, in the instrument-to-arm mapping model, a haptic interface device (HID) indicator with the instrument data, wherein the HID indicator indicates whether a first HID or a second HID of the console is configured to control the instrument.

25-31. (canceled)
